(19) United States
(12) Patent Application Publication
(10) Pub. No.: US 2022/0387201 A1
(43) Pub. Date: Dec. 8, 2022

(54) CATHETER SYSTEM FOR ENGAGEMENT WITH AN IMPLANTED MEDICAL DEVICE

(71) Applicant: Excision Medical, Inc., Malvern, PA (US)

(72) Inventors: David Alexander WOOD, Vancouver (CA); Janarthanan SATHANANTHAN, Vancouver (CA); Greg Alan WALTERS, Exton, PA (US); Philip J. HAARSTAD, Chanhassen, MN (US); Ramji IYER, Plymouth, MN (US)

(73) Assignee: Excision Medical, Inc., Malvern, PA (US)

(21) Appl. No.: 17/778,209

(22) PCT Filed: Nov. 19, 2020
(86) PCT No.: PCT/US2020/061368

§ 371 Date: May 19, 2022

Related U.S. Application Data
(60) Provisional application No. 63/039,510, filed on Jun. 16, 2020.
(60) Provisional application No. 62/937,508, filed on Nov. 19, 2019.

(51) Int. Cl. A61F 2/966; A61M 25/00; A61M 25/01

(52) U.S. Cl. CPC A61F 2/966; A61M 25/0074; A61M 25/0105; A61M 2025/0079; A61M 2025/0004

(57) ABSTRACT

An embodiment of the disclosure is a catheter system configured to be attached to an implanted medical device, such as an implanted valve. The catheter includes an engagement assembly having an elongated member that extends along a central axis, and an engagement member coupled to the elongated member. The engagement assembly has a retracted configuration, where the engagement member is disposed within the channel of the catheter, and an engagement configuration, where the engagement member is disposed outside of the channel and extends outwardly along a direction that is angled with respect to the central axis.

### CROSS-REFERENCE TO RELATED APPLICATIONS

The present application claims priority to and the benefit of U.S. Provisional Application No. 62/937,508, filed Nov. 19, 2019, and U.S. Provisional Application No. 63/039,510, filed on Jun. 16, 2020, the entire contents of which are incorporated by reference into the present application.

### TECHNICAL FIELD

The present disclosure is related to a catheter system, and in particular to a catheter system that engages an implanted medical device.

### BACKGROUND

Severe calcific aortic stenosis can be treated with either surgical aortic valve replacement or transcatheter aortic valve replacement (TAVR). TAVR is a less invasive approach for the treatment of severe aortic stenosis compared to surgery. TAVR was initially only offered to patients with an increased surgical risk. Recent evidence has now shown that TAVR is an effective therapy irrespective of surgical risk. TAVR may become the preferred therapy for most patients with calcific aortic stenosis.

Approximately 50% of patients with severe aortic stenosis may have concomitant coronary artery disease. If a patient undergoes surgery, they will undergo concomitant coronary artery bypass grafting for their coronary artery disease. For patients undergoing TAVR, the optimal management of concomitant coronary artery disease is poorly understood. To date, as the majority of TAVR patients have been high risk, concomitant coronary artery disease (CAD) is often managed medically. However, as TAVR expands to lower risk patients with greater likelihood of longevity, the implications of concomitant coronary artery disease will have greater significance. Of importance, in younger lower risk TAVR patients, there is a greater likelihood that patients may require coronary angiography or percutaneous coronary intervention (PCI) to treat concomitant CAD after TAVR. As TAVR becomes the preferred therapy for treating patients with severe symptomatic aortic stenosis, there will be a significant proportion of patients that will require coronary angiography and PCI.

In patients with a prior TAVR device, the performance of both coronary angiography and PCI can be challenging. Unlike a surgical valve, a transcatheter valve has a metallic frame to which the leaflets of the valve are attached. The frame of the transcatheter heart valve may extend above the ostium of the coronary arteries and therefore potentially obstruct a coronary catheter from being able to engage the coronary arteries. Additionally, the commissural alignment of a transcatheter heart valve (THV) is random and thus the post of the THV may be in front of the coronary ostium which makes catheter engagement extremely challenging. Even if a coronary catheter is able to engage the coronary ostium, there may be insufficient ‘guide support’ to be able to deliver a coronary stent.

### SUMMARY

An embodiment of the disclosure is a catheter system configured to be attached to an implanted medical device. In one example the catheter system is configured to engage an implanted valve in a cardiovascular system. The system includes at least one engagement member configured to engage the implanted valve.

In another embodiment, the catheter system includes a catheter and an engagement assembly. The engagement assembly has an elongated member that extends along a central axis, and an engagement member coupled to the elongated member. The engagement assembly has a retracted configuration, where the engagement member is disposed within the channel of the catheter, and an engagement configuration, where the engagement member is disposed outside of the channel and extends outwardly along a direction that is angled with respect to the central axis.

Another embodiment of the present disclosure includes a catheter system. The catheter system includes a catheter having a proximal end, a distal end, and a channel that extends from the proximal end to the distal end and a central axis. The catheter system includes an engagement member having a retracted configuration, where the engagement member is disposed within the channel of the catheter, and an engagement configuration, where the engagement member is disposed outside of the channel and projects outwardly along a direction that is angled with respect to the central axis.

Another embodiment of the present disclosure is a catheter system. The catheter system includes an engagement member that extends along a central axis. The engagement member is configured to transition between a retracted configuration, where at least a portion of the engagement member extends along the central axis, and an expanded configuration where at least a portion of the engagement member extends outwardly from the central axis along a direction that is angled with respect to the central axis.

Another embodiment of the present disclosure is a method for engaging an implanted medical device, such as a valve, using the catheter system as disclosed herein.

### BRIEF DESCRIPTION OF THE DRAWINGS

The foregoing summary, as well as the following detailed description, will be better understood when read in conjunction with the appended drawings. The drawings show illustrative embodiments of the disclosure. It should be understood, however, that the application is not limited to the precise arrangements and instrumentalities shown.

FIG. 1A is a sectional schematic view of a portion of the cardiovascular system illustrating an implanted transcatheter aortic valve, according to an embodiment of the present disclosure;

FIG. 1B is a perspective view of a short transcatheter aortic valve, according to an embodiment of the present disclosure;

FIG. 2 is a schematic view showing a superior-inferior end of the cardiovascular system and the implanted transcatheter aortic valve shown in FIG. 1A;

FIG. 3 is a plan view of the catheter system according to an embodiment of the present disclosure;

FIG. 4 is a detailed perspective view of a distal end of the catheter system shown in FIG. 3;

FIG. 5 is a sectional view of the distal end of the catheter system shown in FIG. 3;

FIG. 6 is an end view of the distal end of the catheter system shown in FIG. 3;

FIG. 7 illustrates a distal end of the catheter system shown positioned within a frame of the implanted transcatheter aortic valve, according to an embodiment of the present disclosure;

FIG. 8 illustrates the distal end of the catheter system shown positioned within a frame of the implanted transcatheter aortic valve shown in FIG. 7, with an expandable member in an engagement configuration;

FIG. 9 is a side perspective detailed view of the distal end of the catheter system shown in FIG. 8, with the frame removed for clarity of illustration;

FIG. 10 is an end detailed view of the distal end of the catheter system shown in FIG. 8, with the frame removed for clarity of illustration;

FIG. 11 illustrates the distal end of the catheter system shown positioned within a frame of the implanted transcatheter aortic valve shown in FIG. 7, with another expandable member in an engagement configuration;

FIG. 12 is a side perspective detailed view of the distal end of the catheter system shown in FIG. 11, with the frame removed for clarity of illustration;

FIG. 13 illustrates a distal end of the catheter system shown positioned within a frame of the implanted transcatheter aortic valve, with an inner catheter extending in a distal direction relative to the distal end of the catheter shown in FIG. 11;

FIG. 14 is a side perspective detailed view of the distal end of the catheter system shown in FIG. 13, with the inner catheter extending in a distal direction relative to the distal end of the catheter shown in FIG. 11, with the frame removed for clarity of illustration;

FIG. 15 illustrates another catheter system shown engaged with a frame of an implanted transcatheter aortic valve, according to an embodiment of the present disclosure;

FIG. 16 is a plan view of a portion of the catheter system shown in FIG. 3;

FIG. 17 is a partial sectional view of a portion of the catheter system engaged with the frame of the valve as shown in FIG. 15;

FIG. 18 illustrates another catheter system shown engaged with a frame of an implanted transcatheter aortic valve, illustrating the distal tip advance toward a coronary ostium;

FIG. 19 is a plan view of a portion of the catheter system shown in FIG. 3 and FIG. 18;

FIG. 20 is a partial sectional view of a portion of the catheter system engaged with the frame of the valve as shown in FIG. 18;

FIG. 21 is an exploded plan view of a catheter system according to another embodiment of the present disclosure;

FIG. 22 is a plan view of an engagement assembly of the catheter system shown in FIG. 21, showing the engagement member in a retracted configuration;

FIG. 23 is a plan view of an engagement assembly of the catheter system shown in FIG. 21, showing the engagement member in an engagement configuration;

FIG. 24 is a plan assembled view of the catheter system shown in FIG. 21, showing the engagement member in the engagement configuration;

FIGS. 25, 26, and 27 illustrate different configurations of a distal end of the catheter system shown in FIG. 21, according to an embodiment of the present disclosure;

FIGS. 28, 29, and 30 illustrate different configurations of a distal end of the catheter system shown in FIG. 21, showing the engagement member in a deployed configuration;

FIG. 31 is a plan view of anchoring catheter of the catheter system shown in FIG. 21, illustrating the anchor in a deployed configuration;

FIG. 32 is an end view of the anchoring catheter shown in FIG. 31;

FIG. 33 is an end view of an anchoring catheter according to another embodiment of the present disclosure;

FIG. 34 is an end view of an anchoring catheter according to another embodiment of the present disclosure;

FIG. 35 illustrates a catheter system according to another embodiment of the present disclosure;

FIG. 36 illustrates the catheter system shown in FIG. 35, showing an engagement assembly in an expanded configuration;

FIG. 37 illustrates an engagement assembly shown in FIGS. 35 and 36 but illustrated in a planar form for illustrative purposes.

FIGS. 38 illustrates the catheter system shown inserted inside the frame of the implanted transcatheter aortic valve;

FIGS. 39 illustrates the catheter system shown inserted inside the frame of the implanted transcatheter aortic valve with its distal end deflected toward the coronary ostia;

FIG. 40 illustrates the catheter system shown inserted inside the frame of the implanted transcatheter aortic valve with its distal end deflected toward the coronary ostia and the anchor deployed to engage the frame; and

FIG. 41 illustrates the catheter system shown inserted inside and anchored to the frame of the implanted transcatheter aortic valve, with an inner catheter projecting therefrom toward the coronary ostia;

### DETAILED DESCRIPTION OF ILLUSTRATIVE EMBODIMENTS

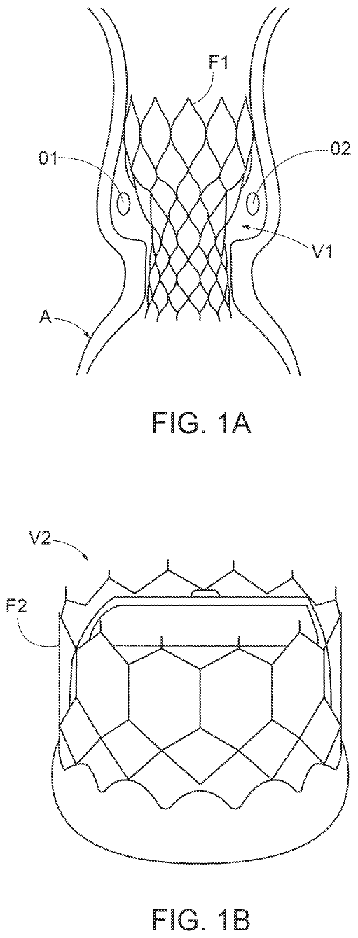

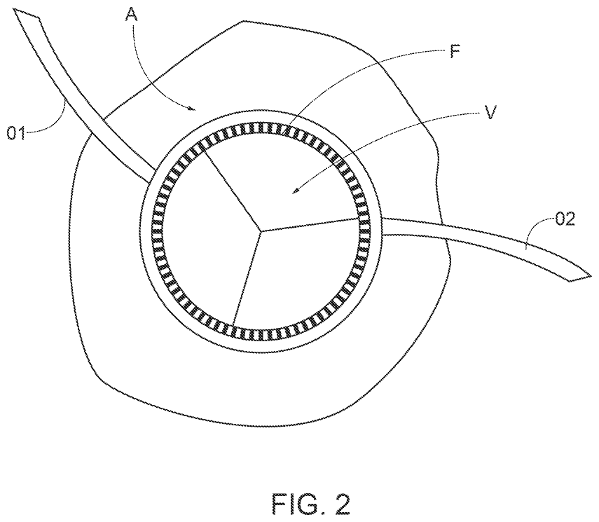

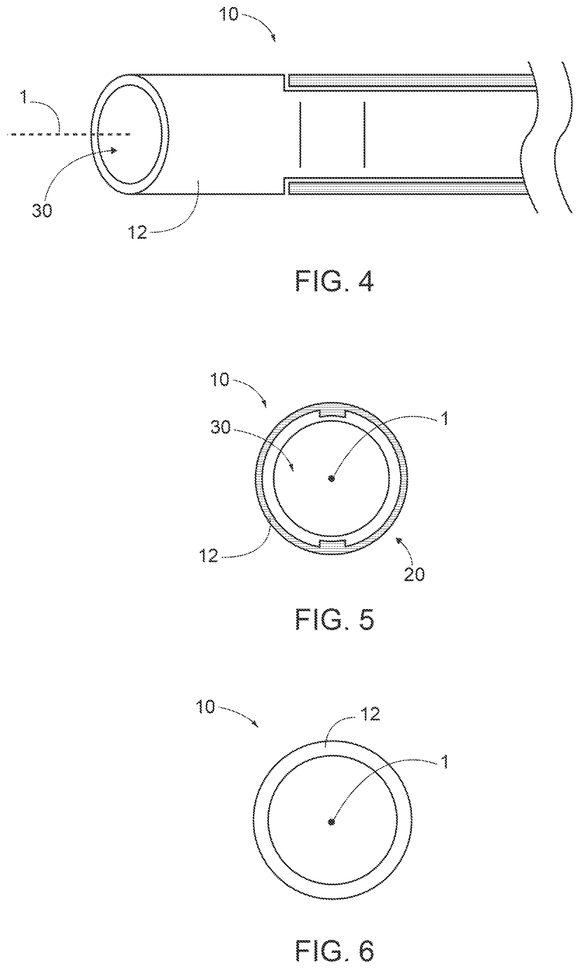

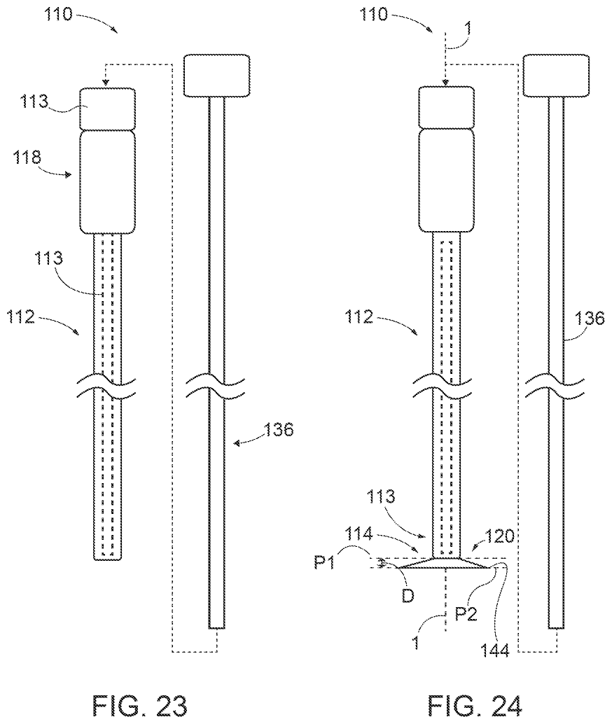

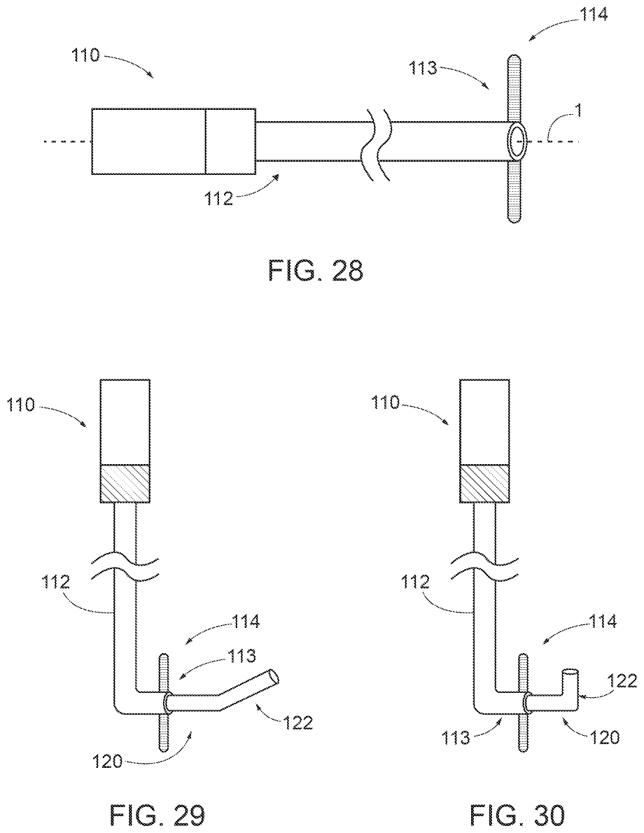

Referring to FIGS. 1A-3, the present disclosure relates to systems, instruments and procedures that permit coupling a guide catheter to an implanted medical device, such as, for example transcatheter heart valve V1 (FIG. 1A) or valve V2 (FIG. 1B). In an exemplary embodiment, the systems, instruments and procedures disclosed here provide access to coronary arteries to perform coronary angiography or percutaneous coronary intervention in patients who have had a prior transcatheter aortic valve implantation or valve replacement in an aorta A. Embodiments of the present disclosure include a catheter system 10 that is configured to permit attachment of a guide catheter 12 to a frame F1 of a transcatheter heart valve (V1, V2) to allow guide support and ease of access to the coronary ostia O1, O2 and arteries. Embodiments of the present disclosure address the problem of challenging coronary ostium engagement and inadequate guide support by disclosing a fully percutaneous coronary catheter system for the performance of coronary angiography and percutaneous coronary intervention, that engages to the frame of the transcatheter heart valve. Various embodiments of engagement mechanisms included in such catheter systems are disclosed and illustrated in FIGS. 1-41.

The catheter system 10 as described may be attached to wide range of valve structures. For instance, a tall valve V1, as shown in FIG. 1A, may be used. In another example, a compact valve V2, as shown in FIG. 1B, may be used. In this disclosure, the reference number V1, V2, and V can all be used interchangeably to refer a valve unless stated otherwise. Exemplary valves include, but are not limited to 1) THV with a tall frame and supra-annular leaflets, e.g. CoreValve Evolut R system, 2) THV with a tall frame and intra-annular leaflets, e.g. the Portico valve, 3) THV with a short frame and intra-annular leaflets, e.g. the SAPIEN 3 valve, and 4) THV with a short frame and supra-annular leaflets, e.g. the Meridian valve.

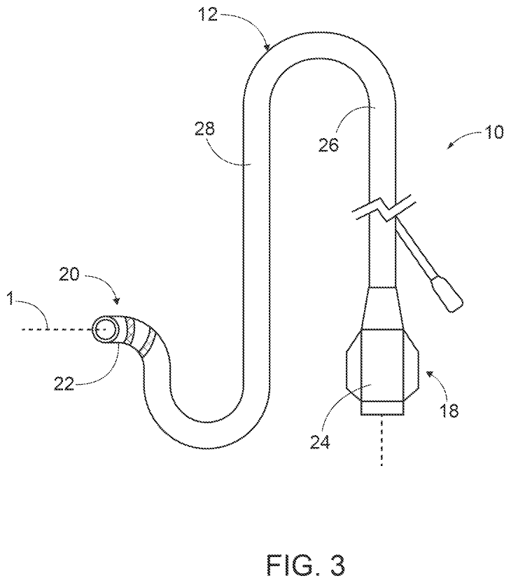

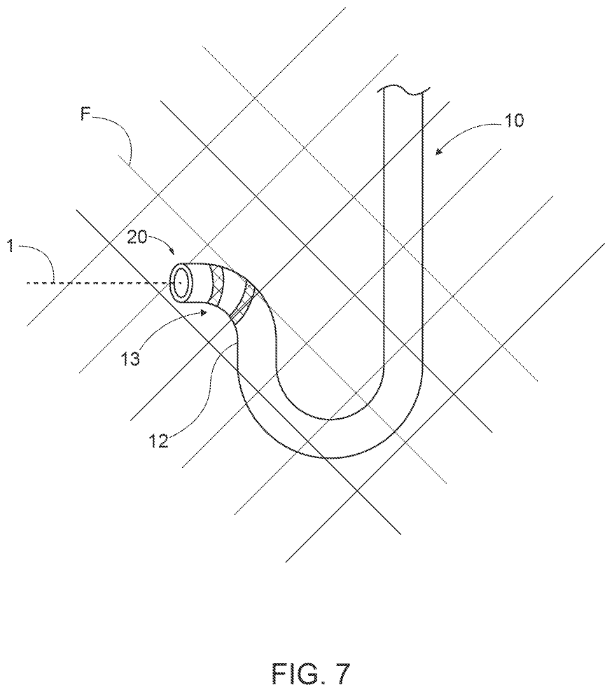

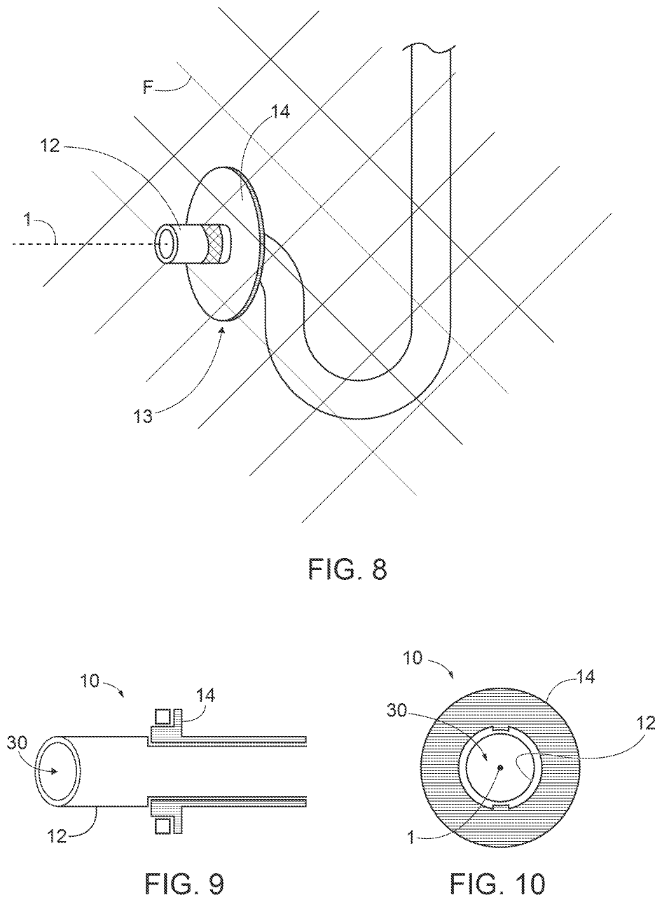

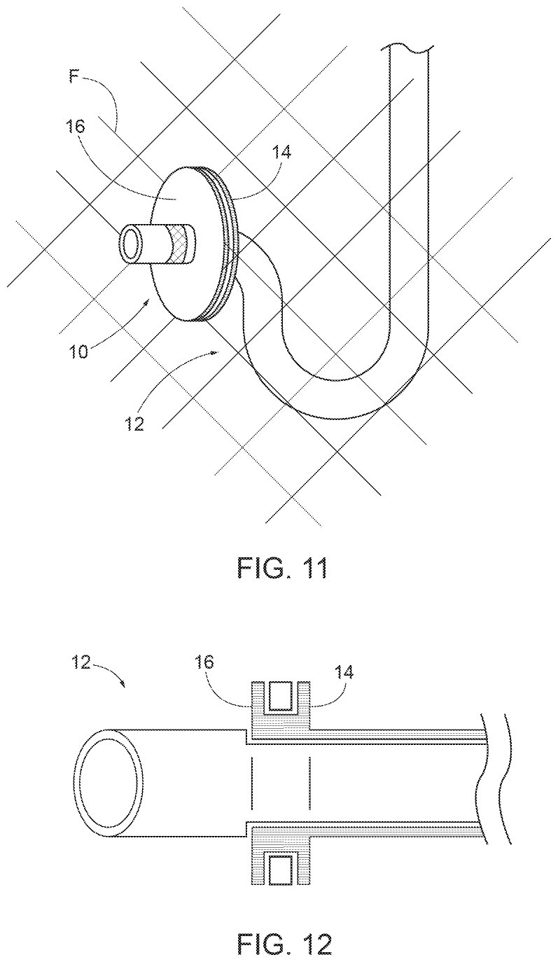

Referring to FIGS. 3-14, the catheter system 10 includes a guide catheter 12 and one or more engagement assemblies 13 (FIGS. 7 and 8). The guide catheter 12 may include a proximal end 18 and a distal end 20 spaced from the proximal end 18 along a central axis 1. The distal end 20 defines a distal tip 22, which may be configured to facilitate engagement of the coronary ostium O1, O2 (ostium shown in FIG. 1). In some examples, the catheter 12 may have one or more radiopaque markers (not numbered) to facilitate position identification during a procedure.

The guide catheter 12 may include a hub 24 at its proximal end and an elongated body 26 coupled to the hub 24. The elongated body 26 includes a shaft 28, a secondary curve, a primary curve, one or more radiopaque markers (not numbered), and the distal tip 22. In cross-section, the catheter 12 may include an inner liner, a middle reinforcing layer (e.g. a braid), and an outer layer or outer jacket. The elongated body 26 defines an inner channel 30 that extends from the distal end 20 toward the proximal end 18 and is sized to receive devices therethrough. The guide catheter may be configured as a steerable catheter. More specifically, the catheter is configured to transition in response to operator input to assume different degrees of flexion of the distal tip 22 to account for different patient anatomy. It should be appreciated that a wide range of catheter shapes and configurations may be used. For instance, the anchoring guide catheter will be available to different shapes similar to currently available guide catheter shapes, i.e., AL 0.75, AL 1, AL 2, JR4, AR mod, VODA 3, VODA 3.5, VODA 4.

One or more engagement assemblies may be used to secure the catheter to the implanted medical device, such as a frame F of an implanted valve. An engagement assembly 13 as used herein is any device, structure, or feature that functions to attach, engage with or otherwise couple a catheter to the subject medical device. The engagement assembly 13, in turn, may include one or more engagement members 14, 16 (FIGS. 11, 12). For example, each engagement assembly may include a single engagement member or two or more engagement members, e.g. a first engagement member 15 and a second engagement member 16. The engagement assembly 13 has described herein is configured to transition from a retracted configuration, as shown in FIG. 7, where the engagement member 14 is disposed within, toward or closer to the central axis 1 of the catheter 12, and an engagement configuration, shown in FIG. 8, where the engagement member 14 extends outwardly along a direction that is angled with respect to the central axis 1.

In one embodiment of an engagement assembly 13 as shown in FIGS. 7-12, the engagement member 14 is an expandable balloon. In one example, the expandable member 14 may be a single balloon on the outer surface of the guide catheter 12, that when inflated will anchor to the THV frame. The single balloon embodiment may be located at or near the distal end 20 of the catheter 12. In another example, the guide catheter can be anchored to the frame F utilizing two expandable members, e.g. two expandable balloons, one of which is disposed proximally with respect to the other. The two expandable balloons are disposed along an outer surface of the guide catheter at the distal end of the guide catheters. When actuated into the engagement configuration, the two expandable balloons expand outwardly and can engage the inner surface (not numbered) and outer surface (not numbered) of the frame F of the valve V, thereby sandwiching the frame F between them. In the engagement configuration, the expanded balloon in contact with the outer surface of the frame F may be referred to as a distal balloon and the balloon in contact with the inner surface of the frame may be referred to as a proximal balloon.

Furthermore, the two expandable members 14, 16 are configured to be inflated independently. In use, in a patient with an implanted THV, the guide catheter 12 will need to be positioned through a cell of the THV frame F (FIG. 7) with an aim to achieve engagement of the catheter to the coronary ostium (not shown). The catheter will be positioned across the THV cell utilizing fluoroscopic guidance and standard percutaneous techniques for the engagement of coronary arteries. Once the catheter 12 is across the cell of a frame F, the engagement assembly 13 will be deployed. “Deployed” means when the engagement assembly has transitioned from the retracted configuration (FIG. 7) into engagement configuration (FIGS. 8 and 11). The position of the expandable members will be evident to the operator on fluoroscopy by two radiopaque markers on the guide catheter 12. The operator will aim to position the two markers on either side of the frame F. Once achieved, the proximal balloon will be expanded first. Then, the distal balloon will then be expanded, and the guide catheter will be anchored to the frame of the THV.

In alternative embodiment, engagement member may be an expandable foam disposed at the distal end of the guide catheter. In such an embodiment, the guide catheter will be able to anchor to the frame of THV utilizing the expandable foam that will be able to conform to the metallic cell of the THV frame and provide anchoring of the guide catheter. The foam will be able to be expanded and deflated.

In another embodiment, the engagement assembly may have at least two engagement members, such as at least two deflectable hooks (not shown). The two (or more) deflectable hooks are configured to be deployed by the operator. The two deflectable hooks, when actuated, attach to the frame F of the THV and anchor the guide catheter.

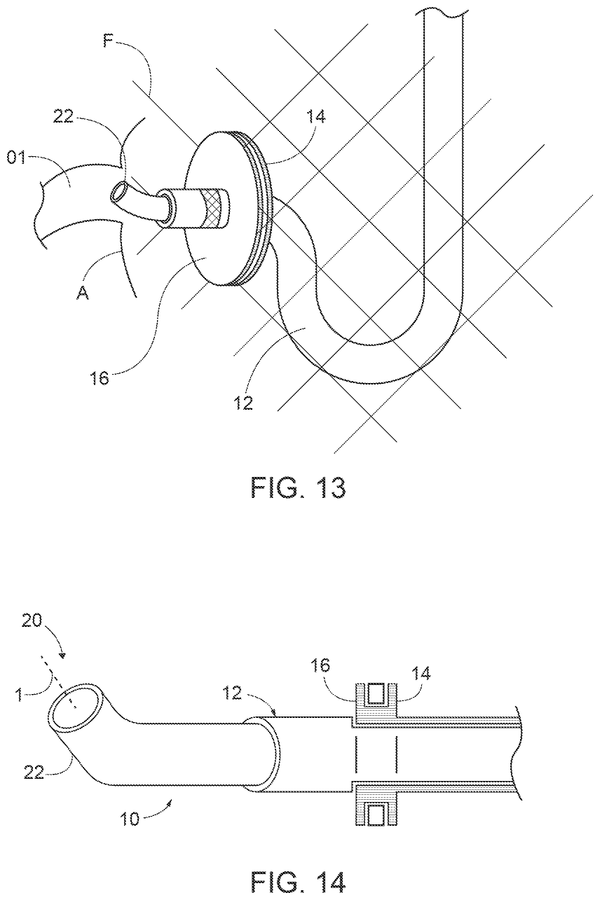

The guide catheter 12 may be configured to facilitate engagement of the coronary ostium. In an embodiment as shown in FIGS. 13 and 14, the distal tip 22 of the catheter will be able to be deflected to allow better alignment with the catheter 12 with the ostium 01 of the coronary artery. In one example, the distal catheter tip of the device may be either a soft-atraumatic tip, or a rigid hard tip. In another example, the distal catheter tip may be deflectable. The distal catheter tip 22 will be able to be deflected in different directions to gain better orientation to the coronary ostium. Deflection may occur in a 360 degree direction relative to a center axis 1 of the catheter 12. In another embodiment, after the guide catheter 12 is anchored to the frame F, the distal tip 22 of the catheter will be able to be extended distally from the body of the catheter 12, to allow engagement with the coronary ostium.

The catheter system 10 may include an inner catheter movable within the guide catheter 12. The inner channel 30 of the guide catheter 12 is sized and configured to accommodate different shaped inner catheters, that may be advanced through the guide catheter and engagement assembly to allow engagement with the coronary ostium O1. As described herein, the catheter would be able to accommodate an inner catheter that would be configured to extend beyond the distal catheter tip and be able to engage the coronary ostium. The inner catheter would be of different shapes to accommodate different patient anatomy.

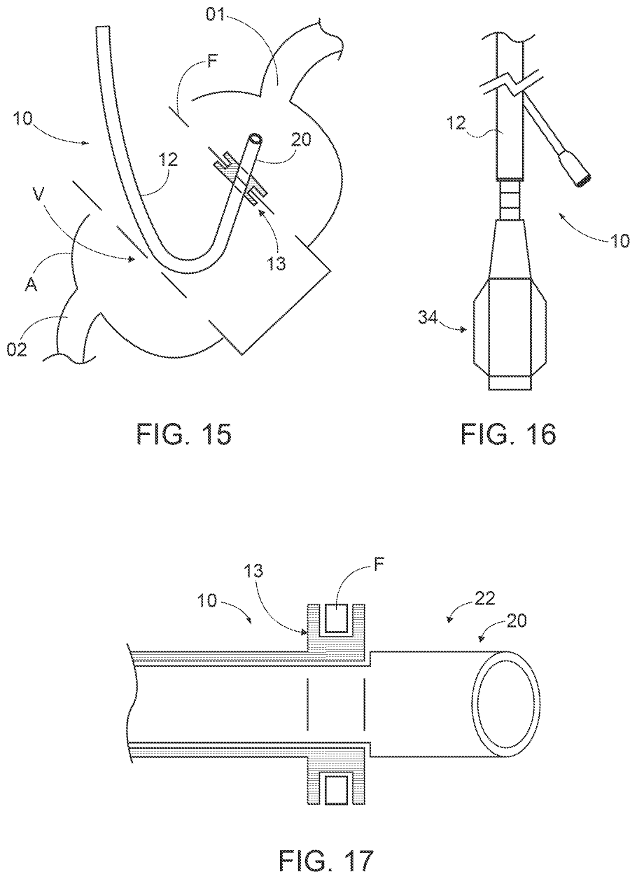

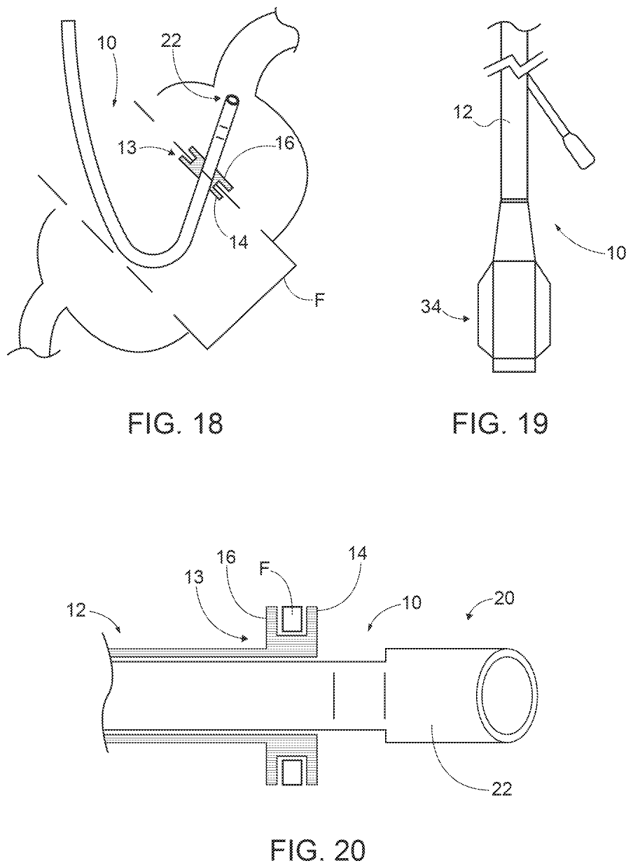

As shown in FIGS. 15-20, the catheter system 10 as described herein may be used in a vascular procedure. For example, the catheter system 10 will be designed to be inserted percutaneously via femoral access or radial access to the aorta A. Arterial access is provided using typical percutaneous techniques whereby a guide wire (not shown) is present in the artery and extends out of the patient. Next, an arterial sheath will be inserted into the artery along the guide wire. The catheter system 10 will be advanced into the artery using an over the wire technique such that a distal end 20 of the guide catheter 12 is placed over the proximal end of the guide wire. The operator will further advance the guide catheter 12 over the wire until a distal end 20 of the guide catheter 12 is proximate the aortic root. The guide catheter 12 will then be positioned through a cell or void of the frame F of the valve V, e.g. to either position its distal end toward the left or right coronary artery, as shown in FIG. 15-17. The catheter system 10 shown in FIG. 19 includes an actuator 34 that is configured to advance the distal tip 22 of the distal end 20 distally from the engagement assembly 13 and frame F, as shown in the transition of distal tip 22 in FIG. 15 (and FIG. 17) relative to FIG. 18 (and FIG. 20). Radiopaque markers on the outside of the distal end 20 of the guide catheter 12 will be visible to the operator using standard catheterization laboratory x-ray equipment. In this manner, the two radiopaque markers will be positioned on either side of the THV frame F. Once the guide catheter 12 is correctly positioned, the engagement assembly 13 will be deployed, as shown in FIGS. 15-20. When the catheter system 10 is anchored to the frame F, the distal end 20 of the guide catheter 12 can be positioned closer toward the desired coronary artery, as shown in FIG. 18. In certain examples, the operator will then perform coronary angiography and percutaneous coronary intervention. After angiography and PCI, the engagement assembly will then be retracted from the frame of the THV. The catheter system 10 will then be pulled back to the ascending aorta removing it from the frame of the THV. The catheter system 10 can then be removed from the body by the operator with or without an over the wire technique.

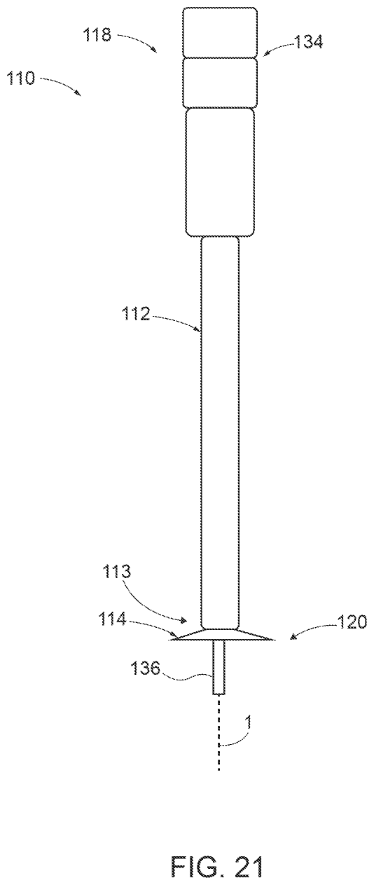

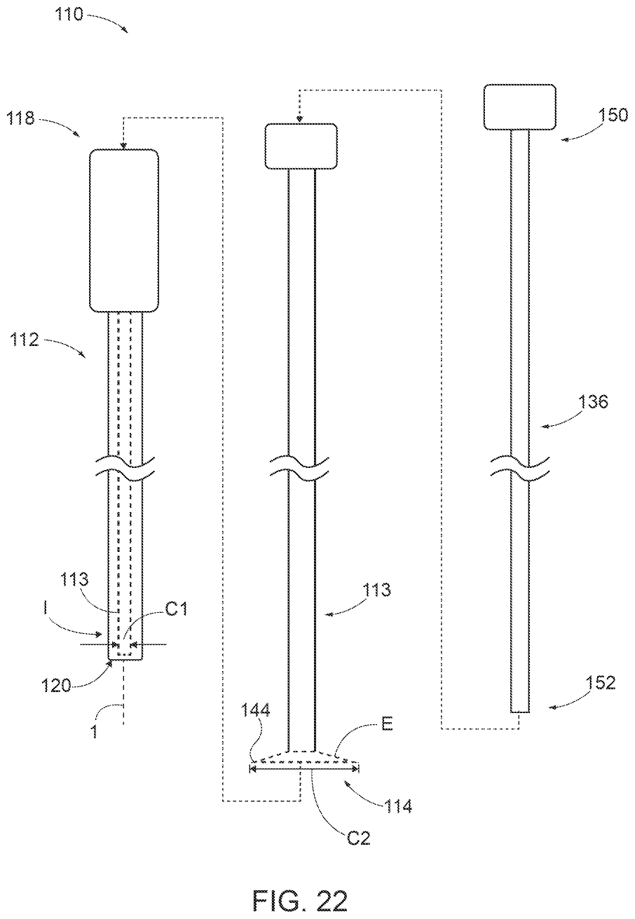

Another embodiment of the present disclosure is illustrated in FIGS. 21-38. FIGS. 21-24 illustrate a catheter system 110 for attaching to a valve V (not shown) implanted in a cardiovascular system. For example, the valve V may be an implanted valve disposed in the aorta of the cardiovascular system. The catheter system 110 may include a catheter 112 and at least one engagement assembly 113 operably coupled to the catheter 112. The engagement assembly 113 includes at least one engagement member 114 that is configured to attach to an implanted frame of a heart valve V. The catheter system 110 may include an inner catheter 136 disposed within the engagement assembly 113. The catheter 112, engagement assembly 113, and inner catheter 136 may be a single unit that is advanced into the aorta as an assembly.

The catheter system 110 may include one or more hubs 134, configured as actuators, at its proximal end 118 configured to operate the catheter system. The actuator is configured to cause transition of the engagement member from the retracted configuration into the engagement configuration. However, in an alternative configuration, each component of the catheter system may be separate devices that are inserted into the aorta in sequence to carry out the functions as described herein. For instance, the catheter may be inserted then the engagement assembly may be inserted into the catheter, etc. Each component of the catheter system is described below.

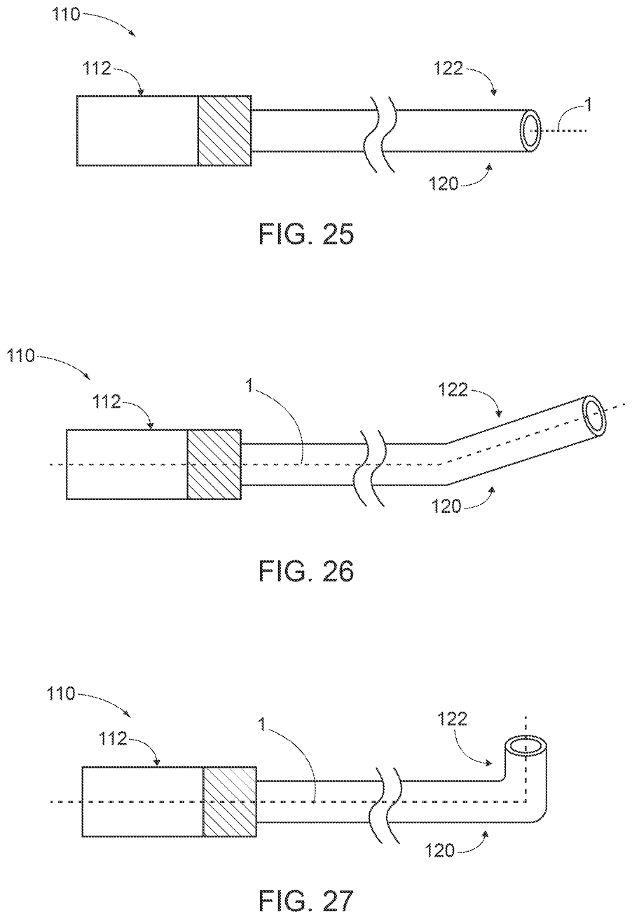

As illustrated, the catheter 112 has a proximal end 118, a distal end 120, and a channel (not shown) that extends from the proximal end 118 to the distal end 120. The channel is configured to receive one or more devices therethrough. For example, the channel can receive and or contain an engagement assembly 113 and an inner catheter 136. The catheter 112 may further comprise at least one marker (not numbered) configured to permit identification of a position of its distal end relative to the valve. The distal end 120 of the catheter 112 is configured to pass through a cellular void of the frame. Furthermore, as shown in FIGS. 25-30, the distal end 120 of the catheter 112 defines a distal tip 122 configured to engage a coronary ostia (not shown). In addition, the distal end of the catheter is either flexible or rigid. In one example, the distal end 120 of the catheter is deflectable in one or more directions that is angularly offset with respect to the central axis 1 of the catheter, as shown in FIGS. 25, 26, and 27. Thus, in an embodiment, the catheter will have a deflectable distal end that would be controlled from the proximal end of the catheter. The distal end will be able to deflect into different shapes to allow passage of the member through the cell of a transcatheter heart valve.

Turning to FIG. 22, in addition to generally expanding upon exit of the catheter, the engagement assembly 113 is configured to radially expand. More specifically, the engagement body defines a terminal outer edge 144, which, in turn, defines A) a first cross-sectional dimension C1 when the engagement assembly is in the retracted configuration I, as shown in FIG. 22, and B) a second cross-sectional dimension C2 that is greater than the first cross-sectional dimension when the engagement assembly is in the engagement configuration, as also shown in FIG. 22. In this example, the second cross-sectional dimension C2 is greater than the first cross-sectional dimension.

Referring to FIG. 24, the distal end 120 of the catheter defines a catheter terminal edge 148 on a first plane P1 that is perpendicular to and intersects the central axis 1 of the engagement assembly 113. The terminal outer edge 144 that lies on a second plane P2 in the engagement configuration. Thus, in the engagement configuration, the terminal outer edge 144 is spaced from the first plane P1 a distance D up to about 2.0 mm. In this example, the distance is parallel to the central axis 1. In one example, the distance D is between 0.25 mm and 2.0 mm.

Continuing FIGS. 21-24, the inner catheter 136 has a proximal end 150, a distal end 152, and an internal channel that extends from the proximal end 150 to the distal end 152. The inner catheter 136 is configured to slide through the internal channel of the engagement assembly 113 such that the distal end 152 advances in a distal direction relative to the distal end of the engagement assembly 113. In one example, the catheter 112 has a first length, the engagement assembly 113 has a second length that is greater than the first length, and the inner catheter 136 has a third length that is greater than the second length. The inner catheter 136 may have a distal end 152 that is flexible or rigid as needed. Furthermore, the inner catheter 136 can be a steerable catheter.

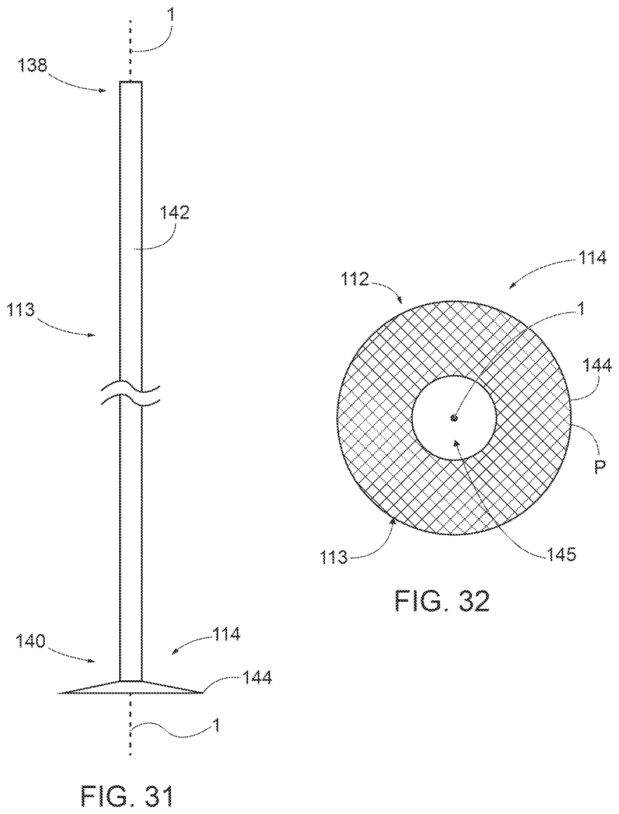

As shown in FIGS. 31-32, the engagement assembly 113 includes a proximal end 138, an engagement end 140, and an inner channel 145 that extends along the central axis 1 from the proximal end 138 to engagement end 140 with the engagement end including the engagement member 114. Furthermore, the engagement assembly 113 includes an elongated member 142 that extends along a central axis 1 and an engagement member 114 coupled to the elongated member 142. The elongated member 142 can have a shaft, tube, or other elongated shape. The engagement member 114 engages, anchors, attaches, or otherwise can be coupled to a frame of a valve. In this regard, the engagement member 114 may be called an anchor. Regardless of the specific type of engagement member, the engagement assembly 113 has a retracted configuration, where the engagement member 114 is disposed toward and along the central axis 1 (e.g. within the channel of the catheter), and an engagement configuration, where the engagement member is disposed outside of the channel and extends outwardly along a direction that is angled with respect to the central axis 1. The engagement member 114 in the engagement configuration is shown in FIGS. 21, 22, 24, 31 and 32.

As shown in the transition of FIGS. 22, 23, and 24, advancement of the engagement assembly 113 is in a distal direction until the engagement member 114 exits the channel 130 of the catheter permits the engagement member to radially expand into the engagement configuration. In the engagement configuration, the engagement assembly 113 is attachable the frame of the valve V (not shown). More specifically, in engagement configuration, the engagement member 114 is disposed outside of the channel and extends outwardly along a radial direction that is substantially perpendicular to the central axis 1 so that engagement member 114 is engageable a frame of the valve. The engagement member 114 is configured to expand adjacent to an interior or exterior surface of the valve to anchor the catheter in place.

In FIGS. 31 and 32, it can be seen that the engagement member may define a terminal outer edge 144 that, in turn, defines a perimeter P. The engagement member 114 can expand such that its perimeter P expands radially relative to the central axis 1 as the engagement assembly 113 transitions from the retracted configuration into the engagement configuration. In one example, the engagement member 114 has a generally tubular shape in the retracted configuration and a generally trumpet shape in the engagement configuration. In this regard, the engagement member may be called an anchor or even a trumpet anchor.

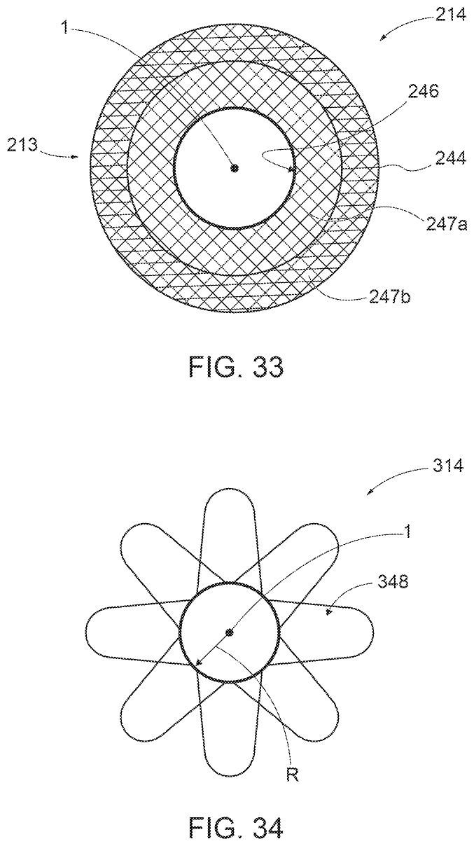

As shown in FIG. 33, an alternative engagement assembly 213 includes an engagement member 214 that defines an engagement body (not numbered) that includes a base 246 disposed at the elongated member and a terminal outer edge 244, and a mass or mass density that varies in a direction from the base 246 toward the terminal outer edge 244. The engagement body may have a first portion 247a with a first mass density and a second portion 247b with a second mass density that is greater than the first mass density. Thus, in this example, the engagement member 214 will have a differential distribution allowing more layers of material at the distal edges of the deployed circular anchor, allowing differential strength. In such an example, engagement member may be a flexible wire mesh configured to expand outwardly. In one example, the flexible wire mesh is comprised of nitinol.

In one alternative embodiment of an engagement assembly shown in FIG. 34, an engagement member 314 or anchor has a base 346 and a plurality of lobes 348 such that in the retracted configuration, the plurality of lobes 348 each extend from the base 346 along the central axis 1. However, in the engagement configuration in such an example, the plurality of lobes 348 flex outwardly along a radial direction R that is substantially perpendicular to the central axis A. In such an embodiment the engagement member 314 or anchor is a laser cut material with a trumpet shape.

The engagement member is configured to articulate into different shapes to allow optimal engagement of the desired cell of the transcatheter heart valve. This will avoid the need for the operator to trial different pre-shaped catheters in order to find the optimal shape to engage the desired THV cell. The trumpet catheter with its unique ability to articulate will allow the operator to change the system into different shapes. The catheter system with its ability to articulate and its anchor mechanism will allow the catheter to avoid any significant interaction with the leaflets of the THV valve. This will allow the THV leaflets to open and close normally, without any interaction with the trumpet catheter.

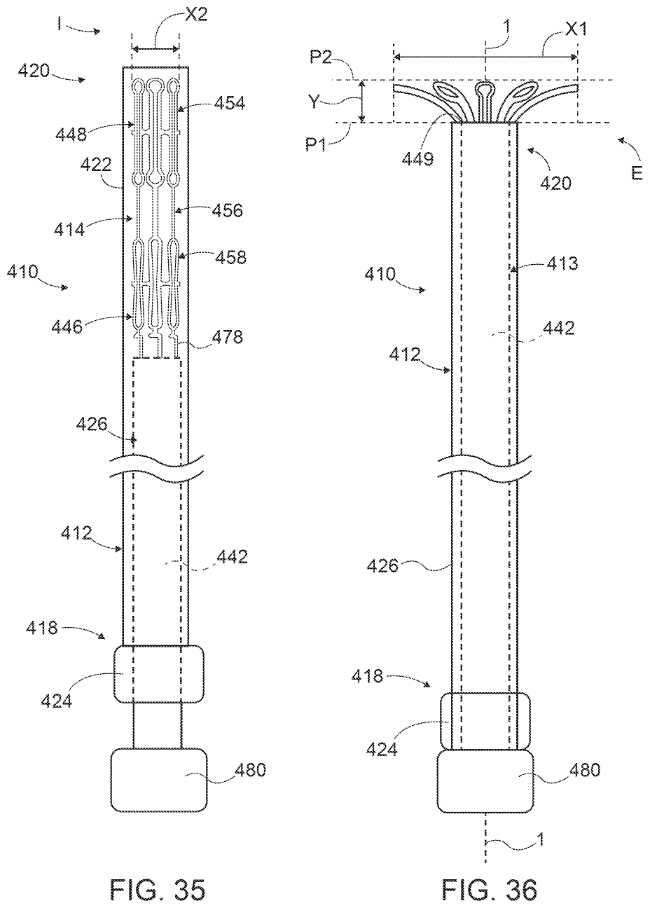

Another embodiment of a catheter system as described herein is illustrated in FIGS. 35-37. As shown in FIGS. 35 and 36, the catheter system 410 includes a catheter 412 and one or more engagement assemblies 413 configured to engage a frame of a medical device, such as an implanted valve. The embodiment of the catheter system 410 is similar to the catheter systems 10, 110, etc. described elsewhere the present disclosure. For instance, the catheter system 410 may include an inner catheter, which has features similar to catheter 136 described above.

The catheter 412 has a proximal end 418, a distal end 420 spaced from the proximal end 418, a channel (not shown) extending therethrough. The channel is sized and configured to receive and allow the engagement assembly 413 to move relative to the catheter 412 along a central axis 1. The catheter 412 may also include a hub 424 at its proximal end 418 and an elongated body 426 coupled to the hub 424. The elongated body 426 may optionally include a shaft, a secondary curve, a primary curve, one or more radiopaque markers (not numbered), and the distal tip 422. In cross-section, the catheter 412 may optionally include an inner liner, a middle reinforcing layer (e.g. a braid), and an outer layer or outer jacket. The catheter 412 may be configured to transition in response to operator input to assume different degrees of flexion of the distal tip 422 to account for different patient anatomy. It should be appreciated that a wide range of catheter shapes and configurations may be used. For instance, the anchoring guide catheter will be available to different shapes similar to currently available guide catheter shapes, i.e., AL 0.75, AL 1, AL 2, JR4, AR mod, VODA 3, VODA 3.5, VODA 4.

As shown in FIG. 35-36, the engagement assembly 413 includes an elongated member 442 and an engagement member 414 coupled to the elongated member 442. The engagement member 414, however, is configured to transition between a retracted configuration I shown in FIG. 35 and an engagement configuration E shown in FIG. 36. The engagement member 414 engages, anchors, attaches, or otherwise can be coupled to a frame of a valve. The elongated member 442 that extends along a central axis 1 and may include a channel that can receive an inner catheter or other devices therethrough. The elongated member 442 can be a shaft, tube, or other elongated shape. The elongated member 442 may include a hub 480 attached to its proximal end (not numbered). The hub 480 may be used by the operator to advance or retract the elongated member, and thus the engagement assembly 413, as needed. Constructed in this way, the elongated member 442 and hub 480 can function as a push-pull rod to control advancement and retraction of the engagement assembly 413. In another embodiment, the hub 480 may be configured as an actuator, actuation of which causes the engagement assembly 413 to advance out of the catheter 412. In such an embodiment, the hub 480 may include levers, slides, guide tracks, rotatable knobs and other mechanisms, that when, operated cause translation of the engagement assembly 413 in the desired direction.

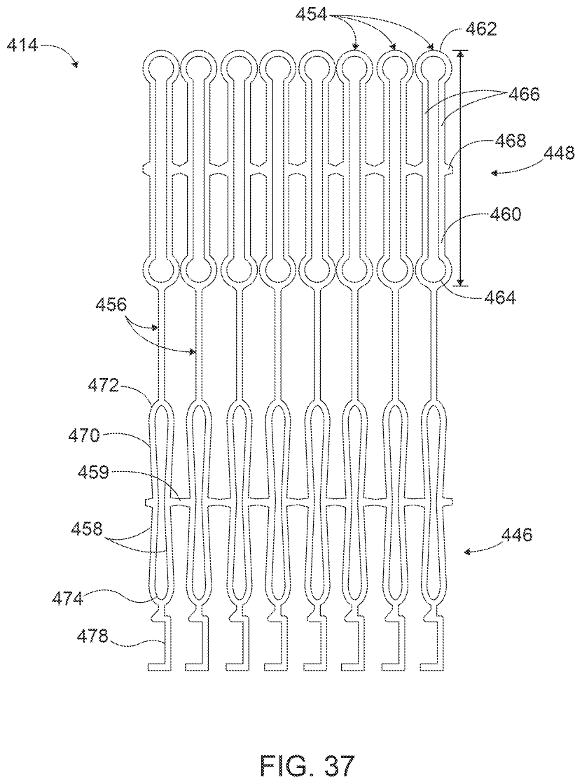

As shown in FIGS. 35-37, the engagement member 414 may include a coupling end 446 and an engagement end 448 and is configured to at least partially expand outwardly to form a means to engage a frame of the valve V. FIG. 37 includes a planer view of the engagement member 414 to illustrate its structure, according to an embodiment of the present disclosure. Thus, what is shown in FIG. 37 is engagement member 414, not in a tubular form for use in the catheter system (as in FIGS. 35 and 36), but unrolled into a planer schematic representation.

In FIGS. 35-37, is can be seen that the engagement assembly 413 may include a plurality of anchoring cells 454, a plurality of longitudinal bridge members 456, and a plurality of catheter coupling cells 458 at the coupling end 446 and that are coupled to the plurality of longitudinal bridge members 456. The engagement assembly 413 extends around a central axis 1 (in use) and each of the plurality of anchoring cells 454 and the plurality of coupling cells 458 extend along a direction that is parallel to the central axis 1. Thus, the engagement assembly 413 has generally tubular shape in the retracted configuration I and an expanded or trumpet shape in the expanded configuration E. The engagement assembly is a laser-cut material and is set in the expanded configuration E during the manufacturing process. During assembly, the engagement assembly is retracted and contained within the catheter 412 so that it is held in the retracted configuration I in the generally tubular shape. As the engagement member 414 exits the catheter 412, the anchoring cells flex outwardly in a direction away from the central axis 1 into engagement configuration or shape.

Continuing with FIG. 37, each anchoring cell 454 includes an anchoring cell body 460 having a leading edge 462, a trailing edge 464, and lateral sides 466 that extends between the leading edge 462 and the trailing edge 464, and a transverse bridge 468 coupled to an adjacent anchoring cell. The anchoring cell 454 has a length F that extends from the leading edge 462 to the trailing edge 464 along a central axis 1. In one example, the length F is between 1.25 mm to about 10 mm In another example, the length is least 1.25 mm In yet another example, the length F is less than or equal to 10 mm. In another example, the length is between 1.25 mm and 7 mm. The lateral sides 466 extend along a direction that is generally parallel to the central axis 1. The lateral sides 466 are flexible and help bias the anchoring cells outwardly when the engagement assembly 413 is advance in a proximal direction out of the catheter 412. Each transverse bridge 468 couples adjacent anchoring cells to each other for added stability.

Continuing with FIG. 37, each coupling cell 458 includes a coupling cell body 470 having a distal edge 472, a proximal edge 474, lateral sides 476 that extend between the distal edge 472 and the proximal edge 474, a transverse bridge 459, and a hook 478 that extends from the proximal edge 474 in a direction away from the distal edge 472. The hook 478 may be used to connect the engagement assembly 413 to the elongated member. Thus, the coupling cells 458 serve as connecting portion between the engagement member 414 and the elongate member.

In the illustrated embodiment, the engagement member 414 may include between one and ten (10) anchoring cells 454, depending on the size of the catheter system 410. It should be appreciated that the engagement member 414 may include more than ten anchoring cells. Furthermore more, the number of anchoring cells 454 and coupling cells 458 generally correspond. The coupling cells may also be formed so that their stiffness is greater or slight greater than the stiffness of the anchoring cells, which can help stabilize the engagement assembly 413 in use.

Referring to FIGS. 35 and 36, the distal end 420 of the catheter defines a catheter terminal edge 449 on a first plane P1 that is perpendicular to and intersects the central axis 1 of the engagement assembly 413. The anchoring cells 454 define a terminal perimeter 444 that lies on a second plane P2 in the engagement configuration. Thus, in the engagement configuration, the terminal outer edge 449 is spaced from the first plane P1 a distance Y up to about 2.0 mm. In this example, the distance Y is parallel to the central axis 1. In one example, the distance D is between 0.25 mm and 2.0 mm.

In use, continuing with FIGS. 35 and 36, movement of the engagement assembly 413 within and relative to the catheter 412 causes the engagement member 414 to transition between a retracted configuration I (FIG. 35), when the engagement assembly 413 is disposed inside the catheter 412, into the expanded configuration E (FIG. 36), where at least portion of the engagement assembly 413 is located outside the catheter 412. As illustrated, the portion of the engagement assembly 413 that exits the catheter 412 includes the anchoring cells 454. With the engagement assembly 413 at least partially located outside the catheter 412, the engagement member 414 expands radially outwardly away from the central axis 1 to engage a frame of the valve (frame and valve not shown). The hub 424 may be used advance to the engagement assembly proximally into the engagement configuration. Furthermore, transition between the insertion and engagement configurations causes the effective cross-sectional dimension of the engagement assembly to transition. For example, the engagement member defines A) a first cross-sectional dimension X1 when the engagement assembly is in the retracted configuration I, as shown in FIG. 35, and B) a second cross-sectional dimension X2 that is greater than the first cross-sectional dimension X1 when the engagement assembly is in the engagement configuration, as also shown in FIG. 36. In this example, the second cross-sectional dimension X2 is greater than the first cross-sectional dimension X2. Furthermore, the second cross-sectional dimension X2 extends between two outermost points of the engagement member 414 when fully expanded, along a line that is perpendicular to and intersects the central axis 1. The extent, or distance, that the engagement assembly 413 must project from the distal end of the catheter 412 to fully expand radially may vary and could be up to about 2.0 mm (or more as needed), as described more fully elsewhere.

Another embodiment of the present disclosure is a method of manufacturing a catheter system. The method includes laser cutting a plurality of anchoring cells, a plurality of longitudinal bridge members, and a plurality of catheter coupling cells into a metal tubular blank to form an engagement assembly. The engagement assembly may be expanded so that the anchoring cells flare outwardly. The method also includes coupling the engagement assembly to a distal end of an elongated member. The anchoring cells are then heat set int this expanded shape. The engagement assembly (and elongated member) is then inserted into the channel of the catheter so that the anchoring cells are pressed inwardly toward the central axis. In this manner, the engagement assembly is retained in the retracted configuration I within the catheter.

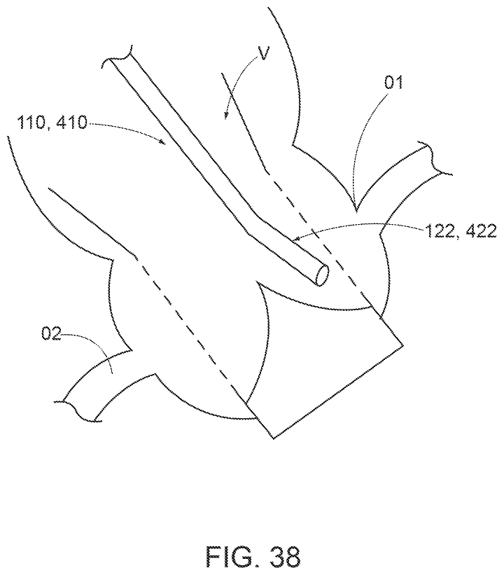

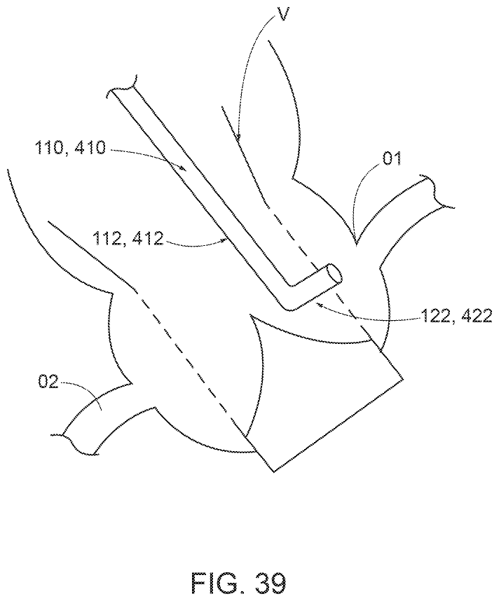

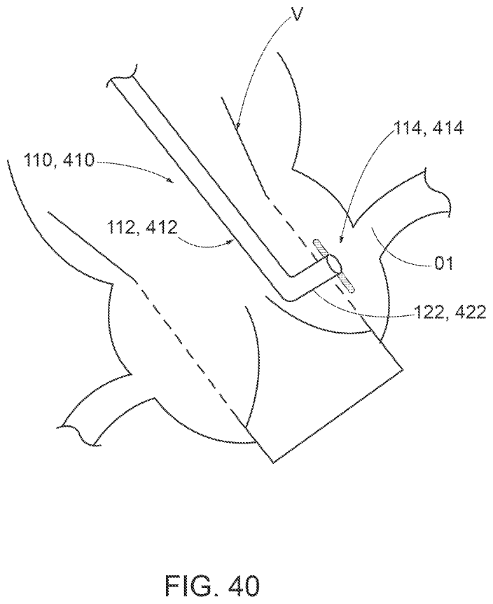

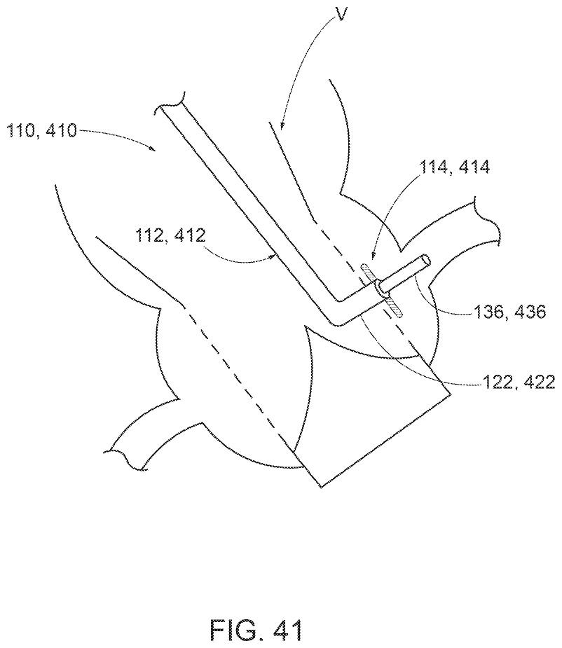

The embodiment described herein may be used during a relevant surgical procedure to couple a catheter to an implanted medical device, as shown FIGS. 38-41. As shown in FIG. 38, a catheter system 110, 410 is advanced to the aortic root and positioned adjacent to the transcatheter heart valve frame V by the desired coronary ostium 01 that the operator wishes to access. As shown in FIGS. 38 and 39, the operator will then be able to deflect the distal tip 122, 422 of the catheter 112, 412 to the desired shape to allow optimum passage of the catheter through the desired cell of the transcatheter heart valve V. Deflection of the distal tip 122, 422 of the catheter 112, 412 would be controlled by the operator from the of the catheter using a specific mechanism, such as a hub, e.g. a push-pull rod or actuator. Once the catheter 112, 412 is through the desired cell of the frame, the operator will then be able to cause the engagement member 114, 414 to transition from the retracted configuration I, shown in FIGS. 38 and 39, into the engagement configuration E (shown in FIGS. 39 and 40). More specifically, the engagement assembly 413 (not shown in FIGS. 38-41) deploys the engagement member into the trumpet shape, which will be located at the distal end of the catheter. Once the engagement member 114, 414 is deployed, an inner catheter 136, 436 can be advanced through the catheter 112,412 to allow access to the coronary ostium O1, as shown in FIG. 41.

In yet another embodiment of the present disclosure, the method may include inserting into an artery a catheter having a proximal end, a distal end opposite the proximal end, and a channel that extends from the proximal end to the distal end. The method may include advancing the catheter through the artery so that its distal end approaches a frame of an implanted transcatheter valve. The method may include causing the distal end to extend through a cell of the frame and causing engagement member to engage with the frame of the implanted transcatheter valve. The method may include accessing a coronary artery through the channel of the catheter with an instrument.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1. A system configured to engage a portion of a heart valve, comprising:
an elongated member that extends along a central axis, the elongated member having a proximal end and a distal engagement end; and
a first engagement member on the engagement end of the elongated member;
a second engagement member on the engagement end of the elongated member, wherein the first engagement member and the second engagement members each have a retracted configuration, where the first and second engagement members are collapsed toward the central axis, and an engagement configuration, where the first and second engagement members expand outwardly with respect to the central axis and engage the portion of the heart valve therebetween.

2.-3. (canceled)

4. The system according to claim 1, wherein each of the first and second engagement members have a generally tubular shape in the retracted configuration and a generally circular cross-sectional trumpet shape in the engagement configuration.

5. The system according to claim 1, wherein each of the first and second engagement members have a base disposed at the elongated member and a terminal outer edge, wherein a mass of the engagement members vary from the base toward the terminal outer edge.

6. (canceled)

7. The catheter system according to claim 1, wherein the engagement member is a flexible wire mesh.

8. The catheter system according to claim 7, wherein the flexible wire mesh comprised of nitinol.

9.-16. (canceled)

17. The system according to claim 1, wherein the elongated member has an inner channel that extends along the central axis from the proximal end toward the engagement end.

18. The system according to claim 17, further comprising:
an inner catheter having a proximal end, a distal end, and an internal channel that extends from the proximal end to the distal end of the inner catheter, wherein the inner catheter is configured to slide through the inner channel of the elongated member so that its distal end is extendable past the engagement end of the elongated member.

19. The system according to claim 18, wherein the elongated member has a first length, and the inner catheter has a second length that is greater than the first length.

20. The system according to claims 1, further comprising an actuator configured to cause transition of the first and second engagement members from the retracted configuration into the engagement configuration.

21. The system according to claim 1, further comprising at least one marker configured to permit identification of its position in a cardiovascular system.

22.-84. (canceled)

85. The system according to claim 1, further comprising a catheter having a proximal end, a distal end, and a channel that extends from the proximal end to the distal end, wherein the first and second engagement members are movable within and relative to the channel of the catheter.

86. The system according to claim 85, wherein the distal end of the catheter is either flexible, rigid, or deflectable.

87. The system according to claim 1, wherein the first and second engagement members are expandable in sequence.

88. The system according to claim 1, wherein the first and second engagement members are expandable substantially simultaneously.

89. The system according to claim 1, wherein the first and second engagement members are independently expandable.

90. The system according to claim 1, wherein the first and second engagement members are first and second expandable balloons.

91. The system according to claim 1, wherein the first and second engagement members comprise a first and second expandable wire mesh, respectively.

92. The system according to claim 1, further comprising a rod push pull rod configured to transition engagement member from the retracted configuration into the engagement configuration.

93. The system according to claim 18, wherein the distal end of the inner catheter is steerable or is usable in conjunction with a steerable catheter.

94. A system configured to engage a portion of a heart valve, comprising:
a steerable catheter having a proximal end, a distal end, and a channel that extends from the proximal end to the distal end, the distal end of the steerable catheter is positionable proximate the portion of the heart valve;
an elongated member movable in the channel of the steerable catheter, the elongated member being elongated along a central axis and having a proximal end, an engagement end, and an inner channel that extends from the proximal end toward the engagement end along the central axis;
a first engagement member on the engagement end of the elongated member; and
a second engagement member on the engagement end of the elongated member, such that the first and second engagement member are movable relative to the steerable catheter, wherein the first engagement member and the second engagement member each have a retracted configuration, where the first and second engagement members are positioned toward the central axis, and an engagement configuration, where the first and second engagement members expand outwardly with respect to the central axis in order to engage the portion of the heart valve when the first and second engagement members are positioned at or near the heart valve.

95. The system of claim 94, wherein the first and second engagement members are independently expandable.

96. The system of claim 94, wherein the first and second engagement members are first and second expandable balloons.

97. The system of claim 94, wherein the first and second engagement members comprise a first and second expandable wire mesh, respectively.

98. The system of claim 94, further comprising a push-pull rod configured to transition engagement member from the retracted configuration into the engagement configuration.

99. The system of claim 94, further comprising a hub at the proximal end of the elongated member, and an actuator, wherein actuation of the actuator causes the first and second engagement members to transition from the retracted configuration into the engagement configuration.

100. The system of claim 94, further comprising an inner catheter having a proximal end, a distal end, and an internal channel that extends from the proximal end to the distal end of the inner catheter, wherein the inner catheter is movable through the inner channel of the elongated member such that its distal end is extendable past the first and second engagement members.

101. The system of claim 100, wherein the distal end of the inner catheter is steerable.
